(19) United States
(12) Patent Application Publication
(10) Pub. No.: US 2023/0172683 A1
(43) Pub. Date: Jun. 8, 2023

(54) SURGICAL IMAGING SYSTEM AND METHOD

(71) Applicant: nSight Surgical Inc., San Francisco, CA (US)

(72) Inventors: Nathaniel Smith, San Francisco, CA (US); Derek Amanatullah, San Francisco, CA (US); Mark Nicholson, San Francisco, CA (US); John Mauriello, San Francisco, CA (US); Noah Davis, San Francisco, CA (US); Colin Willson, San Francisco, CA (US); Alison Rivas, San Francisco, CA (US)

(21) Appl. No.: 18/074,408

(22) Filed: Dec. 2, 2022

Related U.S. Application Data
(60) Provisional application No. 63/285,466, filed on Dec. 2, 2021.
(60) Provisional application No. 63/285,467, filed on Dec. 2, 2021.

(51) Int. Cl. A61B 90/00; G06T 17/00; G06T 7/70; G06V 10/74

(52) U.S. Cl. CPC A61B 90/361; G06T 17/00; G06T 7/70; G06V 10/761; G06T 2207/30241

(57) ABSTRACT

A method includes accessing a three-dimensional representation of a surgical space and based on the three-dimensional representation, for each object in a first constellation of objects: extracting a first location of the object; detecting a first object type of the object; deriving a first surgical status of the object; calculating a first ranking score of the object based on the first object type and the first surgical status; and storing the first location, the first object type, the first surgical status, and the first ranking score in an object container in a set of object containers. The method also includes selecting a first target object at a first time based on a first target ranking score; articulating a mobile camera to locate a first target location of the first target object; and deriving a trajectory of the first target object based on the set of object containers.

### CROSS-REFERENCE TO RELATED APPLICATIONS

This Application claims the benefit of U.S. Provisional Application Nos. 63/285,466, filed on 2 Dec. 2021, and 63/285,467, filed on 2 Dec. 2021, each of which is incorporated in its entirety by this reference.

### TECHNICAL FIELD

This invention relates generally to the field of surgery management and more specifically to a new and useful surgical imaging system and method in the field of surgery management.

### BRIEF DESCRIPTION OF THE FIGURES

FIGS. 1A and 1B are schematic representations of a system;

FIGS. 2A and 2B are a flowchart representation of a method;

FIG. 3 is a flowchart representation of one variation of the method;

FIG. 4 is a schematic representation of one variation of the system and the method;

FIGS. 5A, 5B, and 5C are schematic representations of variations of the system;

FIG. 6 is a schematic representation of one variation of the system;

FIG. 7 is a schematic representation of one variation of the system; and

FIG. 8 is a schematic representation of one variation of the system.

### DESCRIPTION OF THE EMBODIMENTS

The following description of embodiments of the invention is not intended to limit the invention to these embodiments but rather to enable a person skilled in the art to make and use this invention. Variations, configurations, implementations, example implementations, and examples described herein are optional and are not exclusive to the variations, configurations, implementations, example implementations, and examples they describe. The invention described herein can include any and all permutations of these variations, configurations, implementations, example implementations, and examples.

### 1. Method

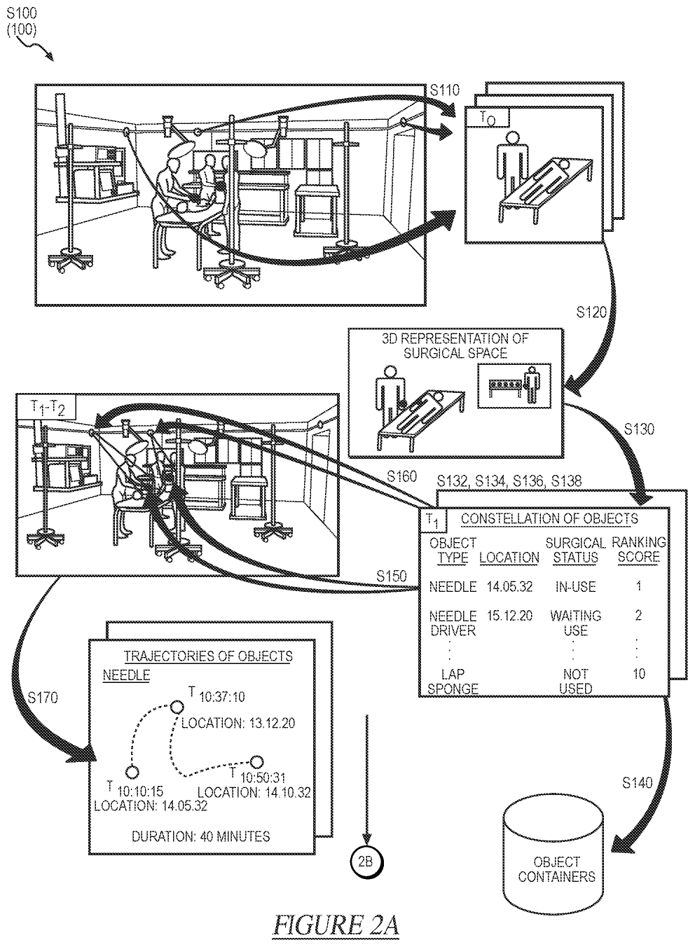

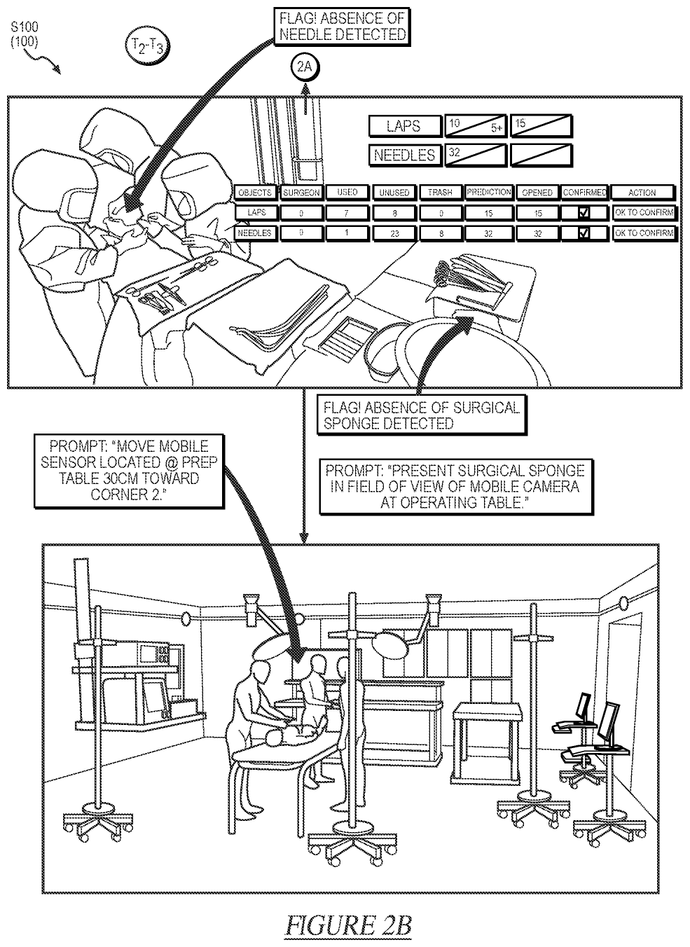

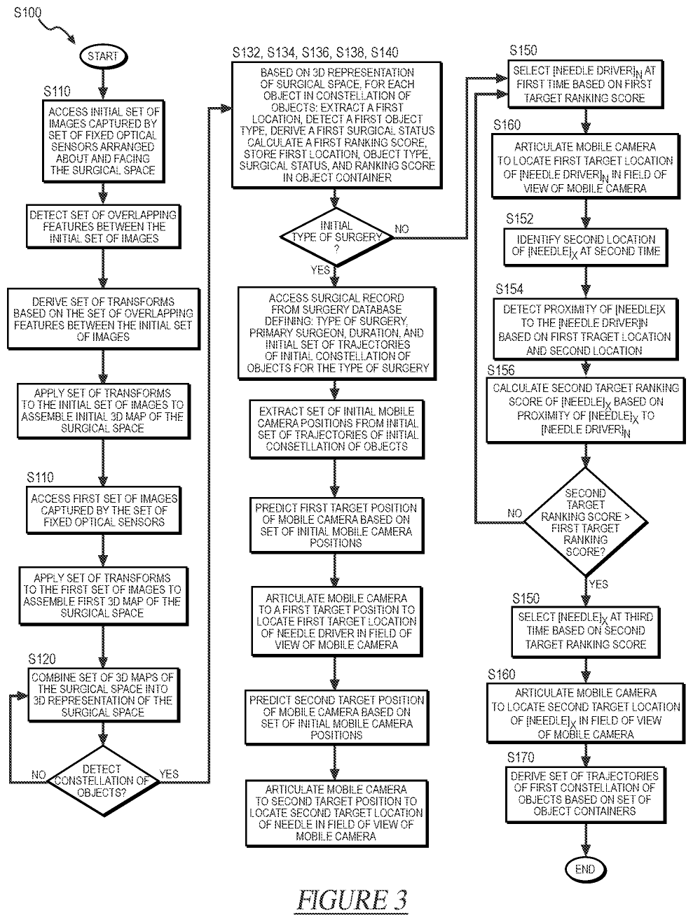

As shown in FIGS. 2A, 2B, and 3, a method S100 for surgical object imaging within a surgical space includes: accessing a first set of images captured by a set of fixed optical sensors arranged about and facing the surgical space in Block S110; aggregating the first set of images into a three-dimensional representation of the surgical space in Block S120; and detecting a first constellation of objects, moving within the surgical space, in the three-dimensional representation in Block S130. The method S100 further includes, based on the three-dimensional representation of the surgical space, for each object in the first constellation of objects: extracting a first location of the object in Block S132; detecting a first object type of the object in Block S134; deriving a first surgical status of the object in Block S136; calculating a first ranking score for the object based on the first object type and the first surgical status in Block S138; and storing the first location, the first object type, the first surgical status, and the first ranking score in an object container in a set of object containers in Block S140. The method S100 also includes: selecting a first target object, in the first constellation of objects, at a first time based on a first target ranking score of the first target object in Block S150; and articulating a mobile camera to locate a first target location of the first target object in a field of view of the mobile camera in Block S160. The method S100 further includes: selecting a second target object, in the first constellation of objects, at a second time succeeding the first time based on a second target ranking score of the second target object, the second target ranking score greater than the first target ranking score of the first target object in Block S150; articulating the mobile camera to locate a second target location of the second target object in the field of view of the mobile camera in Block S160; and deriving a set of trajectories of the first constellation of objects based on object types, locations, surgical statuses, and ranking scores stored in the set of object containers in Block S170.

As shown in FIG. 3, one variation of the method S100 includes, during a first time period, accessing a three-dimensional representation of the surgical space in Block S120. The method S100 also includes, based on the three-dimensional representation of the surgical space, for each object in a first constellation of objects moving within the surgical space: extracting a first location of the object in Block S132; detecting a first object type of the object in Block S134; deriving a first surgical status of the object in Block S136; calculating a first ranking score for the object based on the first object type and the first surgical status in Block S138; and storing the first location, the first object type, the first surgical status, and the first ranking score in an object container in a set of object containers in Block S140. The method S100 further includes: selecting a needle driver, in the first constellation of objects, at a first time based on a first target ranking score of the needle driver in Block S150; and articulating a mobile camera to locate a first target location of the needle driver in a field of view of the mobile camera in Block S160. The method S100 also includes, during a second time period succeeding the first time period: identifying a second location of a needle, in the first constellation of objects, at a second time in Block 152; detecting proximity of the needle to the needle driver based on the first target location and the second location in Block S154; and calculating a second target ranking score of the needle based on proximity of the needle to the needle driver at the third time in Block S156. The method S100 further includes, in response to the second target ranking score of the needle exceeding the first target ranking score of the needle driver: selecting the needle, in the first constellation of objects, at a third time based on the second target ranking score in Block S150; and articulating the mobile camera to locate a second target location of the needle in the field of view of the mobile camera in Block S160.

As shown in FIGS. 2A, 2B, and 3, one variation of the method S100 includes accessing a three-dimensional representation of the surgical space in Block S120, and, based on the three-dimensional representation of the surgical space, for each object in a first constellation of objects moving within the surgical space: extracting a first location of the object in Block S132; detecting a first object type of the object in Block S134; deriving a first surgical status of the object in Block S136; calculating a first ranking score for the object based on the first object type and the first surgical status in Block S138; and storing the first location, the first object type, the first surgical status, and the first ranking score in an object container in a set of object containers in Block S140. The method S100 further includes: selecting a first target object, in the first constellation of objects, at a first time based on a first target ranking score of the first target object in Block S150; articulating a mobile camera to locate a first target location of the first target object in the field of view of the mobile camera in Block S160; in response to detecting absence of the first target object within the field of view of the mobile camera at a second time, articulating a set of fixed optical sensors, arranged about and facing the surgical space, to locate a second target location of the first target object at approximately the second time in Block S162; in response to detecting presence of the first target object within the field of view of the mobile camera at a third time, articulating the mobile camera to locate a third target location of the first target object at approximately the third time in Block S164; and deriving a trajectory of the first target object in a set of trajectories based on images captured by the mobile camera and the set of fixed optical sensors in Block S170.

As shown in FIGS. 2A and 2B, one variation of the method S100 includes: accessing a set of depth images captured by the set of depth sensors located on the perimeter wall of the surgical space by the set of mounting elements and the set of run components in Block S110; fusing the set of depth images into a three-dimensional map of the surgical space in Block S120; detecting a constellation of objects, moving within the surgical space, in the three-dimensional map in Block S130; identifying a first target object, in the constellation of objects, in a field of view of the mobile camera in Block S150; identifying a second target object, in the constellation of objects, in a field of view of the wall-mounted dynamic camera in Block S150; autonomously articulating the mobile camera to track the first target object in the field of view of the mobile camera in Block S160; autonomously articulating the wall-mounted dynamic camera to track the second target object in the field of view of the wall-mounted dynamic camera in Block S160; generating a prompt to reposition the mobile sensor unit in response to loss of the first target object in the field of view of the mobile camera in Block S172; and generating a prompt to present the second target object in response to loss of the second target object in the field of view of the wall-mounted dynamic camera in Block S174.

### 2. Surgical Imaging System

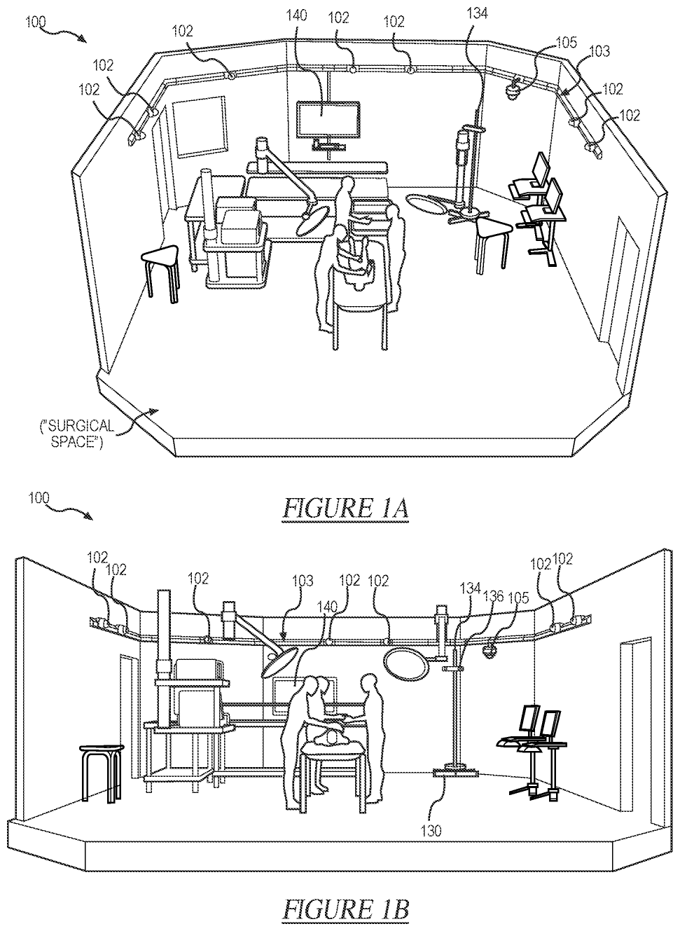

As shown in FIGS. 1A, 1B, 2A, 2B, and 4-8, a surgical imaging system includes: a set of depth sensors 102 (e.g., set of fixed optical sensors); a wall-mounted dynamic camera 105 (e.g., mobile camera); a set of run components 110; a set of run covers 112; a set of turn components 120; a set of turn covers 122; a set of couplers 124; a set of mounting elements 125; a mobile sensor unit 130; and a computer system.

The set of run components 110 is configured to install on a perimeter wall of a surgical space. Each run component defines a linear sweep (or “extrusion”) of a truss cross-section.

Each run cover 112: defines a linear sweep (or “extrusion”) of a cover cross-section; is configured to install over and attach to a run component 110; and cooperates with the run component 110 to a) define a linear interior channel configured to house power and/or data lines, b) define an envelope around the power and/or data lines, c) shed incident dust and liquids downwardly and outside of the envelope, and d) prevent ingress of dust and liquids into the linear interior channel.

Each turn component 120 defines an arcuate (e.g., 90°) sweep of the truss cross-section.

Each turn cover 122: defines an arcuate sweep of the cover cross-section; is configured to install over and attach to a turn component 120; and cooperates with the turn component 120 to a) define an arcuate interior channel configured to house power and/or data lines, b) define an envelope around the power and/or data lines, c) shed incident dust and liquids downwardly and outside of the envelope, and d) prevent ingress of dust and liquids into the arcuate interior channel.

Each coupler 124 is configured to: locate over adjacent, abutting ends of two run 112 and/or turn covers 122; close a seam between the two run 112 and/or turn covers 122; and lock ends of the two run 112 and/or turn covers 122 to the adjacent run 110 and/or turn components 120.

The set of mounting elements 125 is configured to install over the set of run covers 112 and to locate the set of depth sensors 102 and the wall-mounted dynamic camera 105 facing into the surgical space.

The mobile sensor unit 130 is configured for dynamic positioning within the surgical space and includes: a set of wheels 132; a mast supported by the set of wheels 134; and a mobile camera located on the mast 136.

The computer system is configured to execute Blocks of the method S100 described below.

### 3. Applications

Generally, the surgical imaging system (hereinafter the “system 100”) includes a kit of components configured: to locate on a perimeter wall of a surgical space; to mount a set of optical sensors; to route power and data lines from these optical sensors to a computer system (e.g., a server rack) located in the surgical space; and to seal these power and data lines from dust and fluid contamination. For example, this wall-mounted camera subsystem can include: seven depth sensors (e.g., stereoscopic cameras) defining overlapping fields of view and that support low(er)-resolution monitoring of the entire surgical space and tracking objects greater than a minimum size moving throughout the surgical space; and one dynamic camera (e.g., a pan-tilt-zoom color camera, mobile camera) that supports high(er)-resolution tracking of individual objects moving throughout the surgical space. Furthermore, by mounting on the perimeter wall of the surgical space, such as near a ceiling, this wall-mounted camera subsystem can remain inconspicuous and physically unobtrusive to surgical staff working in the surgical space while enabling a comprehensive three-dimensional view of the space and high-resolution tracking of an individual object moving throughout the surgical space at any one time.

The system 100 can include a mobile sensor unit that includes: a wheeled cart with a mast (or “pole”); and a mobile dynamic camera (e.g., a pan-tilt-zoom color camera) mounted to the mast. Accordingly, the mobile sensor unit can be moved within the surgical space, such as before a surgical operation and/or in real-time during the surgical operation: to accommodate surgical staff preferences for human and material flow through the space; to accommodate different combinations or distributions of materials and equipment for surgical operations of different types occurring within the surgical space over time; and responsive to repeated failure of the system 100 to detect target objects in the field of view of the mobile dynamic camera in the mobile sensor unit at its current position in the surgical space during a surgical operation.

Furthermore, the system 100 includes (or is connected to) a computer system—such as a local computer system (e.g., a server rack) located within the surgical space or a remote computer system (e.g., a computer network)—connected to the set of optical sensors via wired connections running through the run and turn components and connected to the mobile sensor unit via a wired or wireless connection.

The system 100 can further include a display, such as mounted to a wall in the surgical space, configured to render prompts and statuses generated by the computer system. Therefore, the wall-mounted depth sensors can cooperate to capture a complete, circumferential view of the surgical space to enable the computer system to globally track many (e.g., all) objects—greater than a minimum size—present and moving within the surgical space during a surgical operation. The wall-mounted dynamic camera can enable the computer system to track small, high-risk target objects—such as surgical needles and lap sponges moving throughout the surgical space over time—with high resolution and spatiotemporal accuracy. Furthermore, the mobile sensor unit is configured for dynamic placement during a surgical operation: to enable the computer system to track small, high-risk target objects in high-risk, high-activity regions in the surgical space (e.g., a prep table and an operating table) over time; to accommodate changing needs of surgical staff working in the surgical space; and to enable surgical staff to quickly correct gaps in scope of vision of the system 100 by relocating (e.g., pushing) the mobile sensor unit to a different position in the surgical space.

Accordingly, the computer system can execute Blocks of the method S100 to: construct a three-dimensional representation of the surgical space; detect and track larger(r) objects in the surgical space based on images (e.g., depth images) captured by the set of depth sensors; calculate a ranking score of these objects based on proximity to a member of surgical staff, proximity to a patient located on an operating table, proximity to a prep table, risk of injury to surgical staff, risk of loss etc.; identify particular target objects in the fields of view of a mobile camera (e.g., wall-mounted dynamic camera, mobile dynamic camera unit) based on the ranking score of the particular object; and return prompts to the surgical space to present a target object to the system 100 when the system 100 loses sight of the target object (e.g., target object absent from a field of view of a mobile camera).

Additionally, the computer system can execute Blocks of the method S100 to: manually guide the surgical staff working within the surgical space to reorient the mobile camera (e.g., wall-mounted dynamic camera, mobile dynamic camera) and/or autonomously reorient the mobile camera to improve tracking of target objects moving throughout a particular region of the space (e.g., over a prep table, over an operating table); dynamically switch between the set of depth sensors and the mobile camera to capture low(er) resolution images of target objects and high(er) resolution images of target objects, based on ranking scores; and derive trajectories of target objects moving throughout the surgical space based on these images. The computer system can then leverage these trajectories to generate pre-operative, real-time, and post-operative metrics (e.g., feedback) for surgical staff working within the surgical space, and thereby enable surgical staff to accurately and repeatably perform surgical operations over a period of time.

### 4. Truss System

As described above and shown in FIGS. 1A, 1B, 2A, 2B, and 4-8, the system 100 includes: a set of run components configured to install on a perimeter wall of a surgical space, each defining a linear sweep (or “extrusion”) of a truss cross-section; and a set of turn components, each defining an arcuate (e.g., 90°) sweep of the truss cross-section.

In one implementation shown in FIGS. 5A, 5B, and 5C, the truss cross-section includes a C-shaped body defining: an upper boss extending upwardly from a top segment of the C-shaped body; a lower boss extending downwardly from a bottom segment of the C-shaped body; and a lip extending upwardly from a distal end of the lower segment of the C-shaped body and configured to retain a set of wires within the center of the C-shaped body. Each run and turn component can also include a sequence of bores or slots arranged on a rear, vertical segment of its C-shaped body and configured to receive a screw, nail, sheetrock anchor, or other fastener to mount the run or turn component to a wall.

As described above, the system 100 also includes: a set of run covers 112, each defining a linear sweep (or “extrusion”) of a cover cross-section and configured to install over and attach to a run component 110; and a set of turn covers 122, each defining an arcuate sweep of the cover cross-section and configured to install over and attach to a turn component 120.

In one implementation shown in FIGS. 5A, 5B, and 5C, the cover cross-section includes: a C-shaped profile configured to seat over the C-shaped body of the truss cross-section and defining; an upper receiver arranged in the upper segment of the C-shaped profile, facing downwardly, and configured to seat over the upper boss of the truss cross-section to retain the upper segment of the C-shaped profile to the upper segment of the adjacent run 110 or turn component 120; and a lower receiver arranged in the lower segment of the C-shaped profile, facing upwardly, and configured to seat over the lower boss of the truss cross-section to retain the lower segment of the C-shaped profile to the lower segment of the adjacent run 110 or turn component 120.

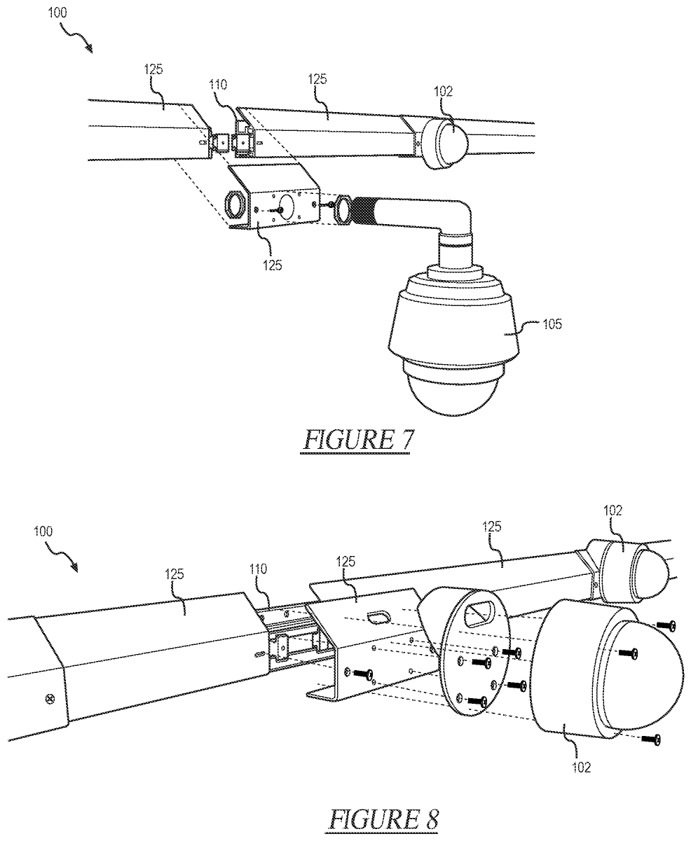

As shown in FIGS. 7 and 8, the system 100 further includes a set of mounting elements 125. In one implementation, a mounting element 125 defines a geometry similar or identical to a cover and further includes a set of bores (or other mounting features): arranged on the vertical segment of its C-shaped body; and configured to mount an optical sensor (e.g., a depth camera, a color camera).

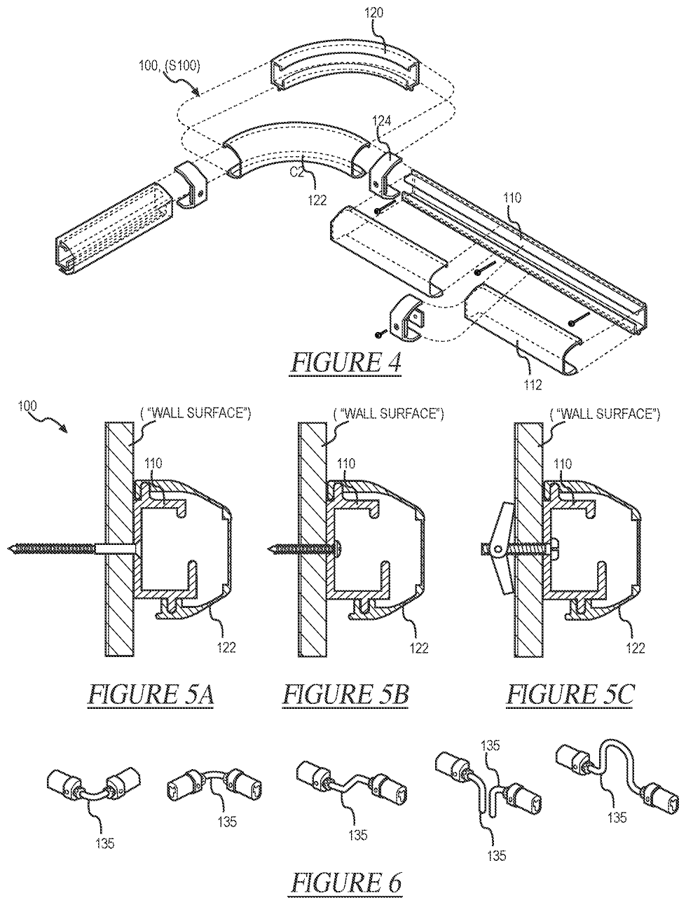

As shown in FIG. 4, the system 100 also includes a set of couplers 124, each configured: to locate over adjacent, abutting ends of two run 112 and/or turn covers 122; to close a seam between the two run 112 and/or turn covers 122; and to lock ends of the two run 112 and/or turn covers 122 to the adjacent run 110 and/or turn components 120.

As shown in FIG. 6, one variation of the system 100 also includes a set of flexible conduit segments 135, each including: a first plug defining a male section configured to insert into an inner channel defined by a run or turn cover installed over a run 110 or turn component 120 and a shoulder inset from the male section, configured to seat inside a coupler, and retained by the coupler; a second plug similar to the first plug; and a flexible conduit (or “tube, pipe”) interposed between the first and second plugs.

### 5. Wall-Mounted Optical Sensors

As shown in FIGS. 1A and 1B, the system 100 can include a set of fixed optical sensors (e.g., wall-mounted optical sensors) arranged about and facing the surgical space and/or mounted to overhead locations on a perimeter wall of the surgical space via mounting elements of the truss system 103.

In one implementation, the system 100 can include: a set of (e.g., seven) fixed optical sensors (e.g., wall-mounted depth sensors); and a smaller quantity of (e.g., one) mobile camera(s) (e.g., wall-mounted dynamic camera). For example, the set of depth images can include a set of stereoscopic cameras defining overlapping fields of view. As described below, the computer system can fuse images (e.g., depth images) captured by these depth sensors into low(er) resolution three-dimensional representations (or “maps”) of the surgical space and then monitor the entire surgical space and track objects—greater than a minimum size—throughout the surgical space based on these three-dimensional maps.

In this example, the mobile camera can include a 2D pan-tilt-zoom color camera. As described below, the computer system can autonomously control the position of the mobile camera (e.g., wall-mounted dynamic camera) to capture high(er)-resolution images of target objects and to track these target objects moving throughout the surgical space during a surgical operation.

### 6. Mobile Sensor Unit

As shown in FIGS. 1A, and 1B, the system wo can include a set of mobile sensor units, each including: a wheeled cart with a mast (e.g., an “IV pole”); and a mobile camera (e.g., mobile dynamic camera) mounted to the mast, such as at a fixed or adjustable overhead position. For example, a mobile camera can include: a 2D pan-tilt-zoom color camera connected to the computer system via a wired or wireless connection; and a power supply (e.g., rechargeable battery).

In one variation, the mobile sensor unit also includes an active or passive optical fiducial, such as a barcode, QR code, or set of light elements. Accordingly, the computer system can detect and track the position and orientation of the wheeled cart—and therefore the mobile sensor unit—within the surgical space (e.g., within a three-dimensional coordinate system assigned to the surgical space) based on the position and orientation of the optical fiducial detected in images captured by the set of fixed optical sensors.

In one example, in preparation for a surgical operation, an operator can locate: a first mobile sensor unit over or facing an operating table; a second mobile sensor unit over or facing a back (or “prep”) table; and a third mobile sensor unit in a back corner of the surgical space. Thus, during the surgical operation, the computer system can: access and process depth images and color images from the fixed optical sensors to track objects moving throughout the surgical space; control and access images from the first and second mobile sensor units to track target objects moving around and between the operating table and the prep table; detect gaps in fields of view of the fixed optical sensors in the surgical space; and prompt surgical staff to move the third mobile sensor unit to target locations within the surgical space to close these gaps and/or decrease frequency of object loss events described below.

### 7. Installation

In one implementation, to install the system 100 in a surgical space, an operator may: locate the computer system (e.g., a server rack) in the surgical space, such as in a corner of an operating room; fasten a series of run components to flat wall surfaces, such as at a height of approximately 84″ above the floor with the first end of the first run component located over the computer system; connect ends of run components with corner sections near corners of the operating room and with flexible conduit segments around other wall features; and connect the first end of the first run component to the computer system via a flexible conduit segment.

The operator may then: install a first power and data line (e.g., Ethernet cable supporting power-over-ethernet data and power connections) from the local computer, into the first end of the first run component, and along the continuous channel defined by the installed run, turn, and flexible conduit sections to the far end of the last run component; install a first optical sensor (e.g., a stereoscopic depth sensor) on a first mounting element; connect the first optical sensor to the first power and data line; install the first mounting element on the far end of the last run component; and install a sequence of run and/or turn covers over installed run and turn components between the first optical sensor and second optical sensor install location.

The operator may then: install a second power and data line from the local computer, into the first end of the first run component, and along the continuous channel defined by the installed run, turn, and flexible conduit sections to the second optical sensor install location; install a second optical sensor on a second mounting element; connect the second optical sensor to the second power and data line; install the second mounting element over an installed run component at the optical sensor install location; and install a sequence of run and/or turn covers over installed run and turn components between the second optical sensor and third optical sensor install location.

The operator can then: repeat this process for a remainder of fixed optical sensors (e.g., wall-mounted optical sensors) allocated for the surgical space; and fasten couplers over each run, turn, and flexible conduit junction to enclose these joints can complete installation of the wall-mounted optical sensors.

In one variation the operator can then: locate a set of mobile sensor units in the surgical space; and connect these mobile sensor units to the computer system via a wired connection or via a wireless communication protocol.

In another variation, the operator can connect a display to the computer system via power and data lines running through the truss system. In one example, the operator can: install a short run component on the wall at a display location and below a junction between two overhead run components; connect the short run component and abutting ends of the two overhead run components via two flexible conduit sections; run display power and data lines from the computer system—through installed run, turn, and flexible condition sections—to this short run component; install a mounting element over the short run component with the display power and data passing through the penetration mount; connect the display power and data lines to a display; and install the display over the short run component and below the overhead run components.

However, the system 100 can be configured to install in a surgical space in any other way.

### 8. Self-Assembly

Once the system 100 is installed within the surgical space with fixed optical sensors (e.g., wall-mounted optical sensors) facing into the surgical space (e.g., toward the center of the surgical space or toward an operating table within the surgical space), the computer system can execute a setup routine to automatically localize the fixed optical sensors and map the surgical space.

### 8.1 Depth Sensor Localization

In one implementation, once the truss system and fixed optical sensors (e.g., wall-mounted optical sensors) are installed in the surgical space, the computer system can: trigger the set of (e.g., seven) depth sensors to capture depth images of the surgical space; detect overlapping features in these depth images; derive a set of transforms that aligns these depth images—and therefore aligns the fields of view of the depth sensors—based on these overlapping features; assemble these depth images into a three-dimensional map of the surgical space; and store this set of transforms to guide future assembly of depth images output by the depth sensors into three-dimensional maps of the surgical space. For example, the computer system can: implement plane fitting, object detection, and/or other computer vision techniques to detect planes (e.g., floors, walls, ceilings) and constellations of objects (e.g., humans, displays, tables, doors, surgical equipment) within these depth images; and derive transforms that align similar planes and similar constellations of similar objects detected in depth images captured by the depth sensors during this setup routine.

In a similar implementation, each optical sensor includes an integrated, outwardly-facing light element that functions as an active fiducial. During the setup routine, the computer system can: trigger a light element in a first optical sensor to activate; triggers all other optical sensors to capture depth images; detect the active light element in these depth images; and generate an initial set of transforms that, when applied to these depth images, aligns the light element—detected in these images—within a three-dimensional representation of the surgical space. In particular, the initial set of transforms represents an initial estimate for positions of the optical sensors within the surgical space. The computer system can then: repeat this process for a second combination of one optical sensor with active light element and other image capture at the remaining optical sensors; detect the active light element in these depth images; refine the set of transforms based on locations of the second light element detected in this next set of depth images; repeat this process for each other combination of one optical sensor with active light element and other image capture at the remaining optical sensors to further refine the set of transforms; and then implement image alignment techniques to further refine the set of transforms to align light elements and other common features (e.g., surfaces, edges, corners) detected around these active light elements in these depth images.

### 8.2 Surgical Space Map: Three-Dimensional Representation of Surgical Space

Therefore, the computer system can: derive a set of transforms that—when applied to a set of depth images captured concurrently by the set of wall-mounted depth sensors (or a set of depth images captured asynchronously by the depth sensors when the surgical space is unoccupied)—align active fiducials and passive features depicted in these depth images; and apply these transforms to a set of concurrent depth images captured by the set of depth sensors—such as during a surgical operation in the surgical space—to assemble a three-dimensional map of fixed (or “immutable”) surfaces, fixed objects, and movable (or “mutable”) objects in the surgical space.

More specifically, during one scan cycle during a surgical operation, the computer system can: trigger the set of depth sensors to capture a concurrent set of depth images; apply the stored transforms for the set of depth sensors to these depth images to generate a three-dimensional map of surfaces within the surgical space; implement object detection, object recognition, and/or other computer vision or computer perception techniques to detect and identify individual objects within the three-dimensional map of the surgical space; and label (or “annotate”) these objects in the three-dimensional map accordingly.

The computer system can then: repeat this process for each subsequent scan cycle to generate one annotated three-dimensional map of the surgical space per scan cycle, such as at a rate of 2 Hz or 60 Hz; and implement object tracking techniques to track individual objects across sequential three-dimensional maps of the surgical space.

Furthermore, the computer system can also store these annotated (and timestamped) three-dimensional maps as a three-dimensional representation of object presence and object flow through the surgical space during the surgical operation.

For example, at an initial time, the computer system can: access an initial set of images captured by the set of fixed optical sensors arranged about and facing the surgical space; detect a set of overlapping features between the initial set of images; derive a set of transforms based on the set of overlapping features between the initial set of images; and apply the set of transforms to the initial set of images to assemble an initial three-dimensional map in a set of three-dimensional maps of the surgical space. Then, the computer system can: access a first set of images captured by the set of fixed optical sensors; apply the set of transforms to the first set of images to assemble a first three-dimensional map in the set of three-dimensional maps of the surgical space; and combine the set of three-dimensional maps of the surgical space into a three-dimensional representation of the surgical space.

### 8.3 Depth Sensor Locations

Furthermore, the transforms that align depth images captured by the depth sensors also represent relative positions of (i.e., linear and angular offsets between) of these wall-mounted depth sensors. Accordingly, the computer system can execute this process to automatically: derive the relative positions of the set of depth sensors within the surgical space (e.g., relative to a coordinate system assigned to the surgical space by the computer system or relative to a first depth sensor in the set); and generate a three-dimensional map of mutable and immutable objects and surfaces within the space.

### 8.4 Mobile Camera: Wall-Mounted Dynamic Camera Calibration

During the setup routine and after generating the set of transforms that represent relative positions of the set of depth images, the computer system can automatically calibrate (or “assemble”) the mobile camera (e.g., wall mounted dynamic camera).

### 8.4.1 No Wall-Mounted Dynamic Camera Fiducial

In one implementation, the computer system: triggers the wall-mounted dynamic camera to enter a first pan, tilt, and zoom position; triggers the wall-mounted dynamic camera to capture a first image (e.g., a 2D color image); implements object recognition techniques such as described above to detect and identify a first set of objects in the first image; extracts a first target constellation of object types (e.g., a map of centroids and/or boundaries of objects of known or predicted types of the first set of object) from the first image; calculates a first plane or a surface (e.g., a semispherical surface) that—when the three-dimensional map of the surgical space is projected onto this first plane or surface—produces a constellation of object types that approximates the first target constellation of object types derived from the first image; and stores this first plane (or surface) in association with the first pan, tilt, and zoom position.

In this implementation, the computer system then: triggers the wall-mounted dynamic camera to enter a second pan, tilt, and zoom position; triggers the wall-mounted dynamic camera to capture a second image; implements object recognition techniques to detect and identify a second set of objects in the second image; extracts a second target constellation of object types from the second image; calculates a second plane or surface that—when the three-dimensional map of the surgical space is projected onto this second plane or surface—produces a constellation of object types that approximates the second target constellation of object types derived from the second image; and stores this second plane (or surface) in association with the second pan, tilt, and zoom position.

The computer system then: repeats this process for other pan, tilt, and zoom positions; and assembles this set of pan, tilt, and zoom positions and the corresponding planes (or surfaces) thus derived into a field of view model that returns a pan, tilt, and maximum zoom position predicted to locate a constellation of objects—at known positions in the three-dimensional of the surgical space—within the field of view of the wall-mounted dynamic camera.

For example, during a scan cycle during a surgical operation, the computer system can: trigger the set of depth sensors to capture a set of concurrent depth images; assemble the set of depth images into a three-dimensional map of the surgical space; detect and/or track a group of objects of interest in the three-dimensional map, such as a hand, forceps, and a lap sponge; derive three-dimensional locations of the group of objects (e.g., a volumetric centroid of each object in the group or three-dimensional points on a smallest surface that fully contains the group of objects) from the current three-dimensional map of the surgical space; and input the three-dimensional locations of the group of objects into the field of view model to calculate a pan, tilt, and maximum zoom position of the dynamic camera that locates the group of objects fully in the field of view of the dynamic camera. The computer system can then drive the dynamic camera to this pan, tilt, and zoom position and implement object detection and object tracking techniques to detect and track the group of objects in a 2D image subsequently captured by the dynamic camera.

During subsequent scan cycles, the computer system can: implement object tracking techniques to modify the pan, tilt, and zoom position of dynamic camera to track the group of objects; track all objects in the three-dimensional map of the surgical space generated from subsequent groups of concurrent depth images captured by the set of depth sensors; and characterize risk or relevance of individual or groups of objects detected in the three-dimensional map. When the computer system identifies an alternate group of objects of (greater) interest, the computer system can execute the foregoing process to reorient the dynamic camera to image and track this alternate group of objects.

8.4.2 Wall-Mounted Dynamic Camera with Fiducial

In another implementation, the wall-mounted dynamic camera includes an active optical fiducial (e.g., a light element). In this implementation, the computer system can: selectively activate this active fiducial when triggering the depth images to capture depth images of the surgical space, such as during the setup routine and/or during a surgical operation; detect the active fiducial in these depth images; estimate the position of the wall-mounted dynamic camera within the three-dimensional map of the surgical space based on the position of the active fiducial detected in these depth images; and define a cone of actuation extending into the surgical space from the estimated position of the wall-mounted dynamic camera within the three-dimensional map of the surgical space based on pan and tilt ranges of the dynamic camera.

The computer system can then: set a zoom position of the wall-mounted dynamic camera; trigger the wall-mounted dynamic camera to enter a first pan and tilt position; trigger the wall-mounted dynamic camera to capture a first image; implement object recognition techniques to detect and identify a first set of objects in the first image; extract a first target constellation of object types from the first image; calculate a first ray—extending from the estimated position of the wall-mounted dynamic camera within the three-dimensional map of the surgical space and bounded by the cone of actuation—such that projection of objects in the three-dimensional map of the surgical space onto a first plane normal to the first ray produces a constellation of object types that approximates the first target constellation of object types derived from the first image with least positional error between the constellation of object types and the target constellation of object types nearest the ray; and store the first ray in association with the first pan and tilt position.

The computer system can then: repeat for other pan and tilt positions; and assemble the set of pan and tilt positions and the corresponding rays into a field of view model that outputs a pan and tilt position of the dynamic camera that locates the focal axis of the dynamic camera to intersect a target position within the cone of actuation defined in the three-dimensional map of the surgical space.

For example, during a scan cycle during a surgical operation, the computer system can: trigger the set of depth sensors to capture a set of concurrent depth images; assemble the set of depth images into a three-dimensional map of the surgical space; detect and/or track a target object of interest in the three-dimensional map, such as a needle driver, which may be loaded with a surgical needle; derive a three-dimensional location of the target object (e.g., a volumetric centroid of the target object) from the three-dimensional map of the surgical space; and input the three-dimensional location of the target object into the field of view model to calculate a pan and tilt position of the dynamic camera that locates the focal axis of the dynamic camera on the target object. The computer system can then drive the dynamic camera to this pan and tilt zoom position; and implement object detection and object tracking techniques to scan subsequent 2D images for the target object and incrementally increase the zoom setting of the dynamic camera until the computer system detects and identifies the target object in this sequence of images with at least a minimum confidence.

During subsequent scan cycles, the computer system can: implement object tracking techniques to modify the pan, tilt, and zoom position of the dynamic camera to track the target object; track all objects in three-dimensional map of the surgical space generated from subsequent groups of concurrent depth images captured by the set of depth sensors; and characterize risk or relevance of individual objects detected in the three-dimensional map. Then, when the computer system identifies an alternate target object of (greater) interest, the computer system can execute the foregoing process to reorient the dynamic camera to image and track this alternate target object.

### 9. Mobile Camera: Mobile Sensor Unit Calibration

The computer system can implement similar methods and techniques to calibrate the mobile sensor unit once placed in the surgical space and activated, such as at the commencement of a surgical operation or after the mobile sensor unit is moved during the surgical operation.

In one implementation, the computer system: scans a current three-dimensional map of the surgical space—generated based on a last set of depth images captured by the depth sensors—for a known set of active or passive fiducials arranged on the mobile sensor unit; and derives a location and orientation of the mobile sensor unit—and specifically a location and orientation of the mobile camera arranged on the mast of the mobile sensor unit—within the surgical space based on the location of these known fiducials detected in the three-dimensional map.

The computer system then accesses a last image captured by the mobile sensor unit and implements methods and techniques described above to: derive a position of a focal axis of the mobile camera in the mobile sensor unit based on positions of a group of object types detected in the image and locations of a similar constellation of object types in the three-dimensional map of the surgical space; and/or derive a field of view model that returns a pan, tilt, and/or maximum zoom position predicted to locate a constellation of objects—at known positions in the three-dimensional map of the surgical space—within the field of view of the mobile camera of the mobile sensor unit.

The computer system can also execute this process to (re)calibrate the position of the mobile sensor unit within the surgical space during a setup routine, prior to or at the commencement of a surgical operation, during a surgical operation in response to detecting motion of the mobile sensor unit, and intermittently during the surgical operation to verify calibration of the mobile sensor unit.

### 10. Intra-Operative Object Tracking

The computer system can then execute the method S100 to track many objects (e.g., most, all large objects) moving within the surgical space at a low(er) resolution and individual target objects (e.g., small, high-risk objects) at a high(er) resolution via images captured by the wall-mounted and mobile optical sensors. In particular, the computer system can execute the foregoing methods and techniques to: generate timeseries three-dimensional maps of the surgical space—representing static and moving objects within the surgical space—based on depth images captured by the set of depth sensors; track a single target object or single target group of objects moving throughout the surgical space at any instant in time based on images captured by the wall-mounted dynamic camera; and track objects within a particular or narrow area of interest within the surgical space around a mobile sensor unit, such as positioned over a prep table or facing the operating table.

In one implementation, during a scan cycle, the computer system: triggers the set of depth sensors to capture a set of concurrent depth images; and assembles this set of concurrent depth images into a three-dimensional map of the surgical space according to the set of transforms generated during the setup routine and stored for the depth sensors. The computer system then implements object detection and/or recognition techniques to identify individual objects in this three-dimensional map, such as including: hands of human walking from prep table toward operating table; a tool in hands of primary surgeon at operating table; a cart containing surgical material entering surgical space; a lap sponge falling within a threshold distance of and moving toward the operating table; and/or a lap sponge in contact with and/or moving away from a patient on the operating table; etc. The computer system then ranks these objects, such as based on: risk of injury to surgical staff; risk of loss in the patient; object type; object proximity to surgical staff; object proximity to the patient; and/or relevance of the object to a current stage or step of the surgical operation. From this set of ranked objects, the computer system selects a first, highest-ranking object that intersects the cone of actuation of the mobile sensor unit. From the remaining set of ranked objects, the computer system then selects a second, highest-ranking object that intersects the cone of actuation of the wall-mounted dynamic camera.

The computer system then: extracts a first 3D location of the first object of interest and a second 3D location of the second object of interest from the three-dimensional map of surgical space; passes the first 3D location into the field of view model of the mobile sensor unit to calculate a first pan, tilt, and/or zoom position that locates the first target object in the field of view of the mobile sensor unit; and passes the second 3D location into the field of view model of the wall-mounted dynamic camera to calculate a second pan, tilt, and/or zoom position that locates the second target object in the field of view of the wall-mounted dynamic camera.

Accordingly, the computer system: drives the mobile sensor unit to the first pan, tilt, and/or zoom position; and implements object detection, recognition, and/or tracking techniques to identify and track the first target object in a stream of images captured by the mobile sensor unit. The computer system can also implement closed-loop controls to adjust the zoom level of the mobile sensor unit to fill the first target object in the field of view of the mobile sensor unit.

Similarly, the computer system: drives the wall-mounted dynamic camera to the second pan, tilt, and/or zoom position; and implements object detection, recognition, and/or tracking techniques to identify and track the second target object in a stream of images captured by the wall-mounted dynamic camera. The computer system can also implement closed-loop controls to adjust the zoom level of the wall-mounted dynamic camera to fill the second target object in the field of view of the wall-mounted dynamic camera.

The computer system can repeat this process for each subsequent scan cycle to: generate a new three-dimensional map of the surgical space based on depth images captured by the set of depth sensors; detect, identify and characterize objects in the three-dimensional map; select target objects in cones of actuation of the mobile sensor unit and the wall-mounted dynamic camera; derive pan, tilt, and/or zoom positions of the mobile sensor unit and the wall-mounted dynamic camera that locate the target objects in corresponding fields of view; and drive the mobile sensor unit and the wall-mounted dynamic camera to these pan, tilt, and zoom positions.

In one variation, when a highest-ranked object in the surgical space is located within but approaches the edge of the cone of actuation of the mobile sensor unit, the computer system can: reallocate tracking of this target object to the wall-mounted dynamic camera; identify a next highest-ranking object that intersects the cone of actuation of the mobile sensor unit; and execute the foregoing methods and techniques to locate the first target object in the field of view of the wall-mounted dynamic camera and to locate the second target object in the field of view of the mobile sensor unit.

### 10.1 Object Ranking+Camera Articulation

In one implementation, during a scan cycle, the computer system can: trigger the set of fixed optical sensors to capture a set of images; and aggregate the set of images into a three-dimensional representation of the surgical space according to the set of transforms generated during the setup routine (e.g., setup period) and stored for the set of fixed optical sensors. The computer system can then implement object detection and/or recognition techniques to: identify a first constellation of objects, moving within the surgical space, in this three-dimensional representation; calculate a ranking score of each object in the first constellation of objects; and articulate a mobile camera to locate a target object associated with the highest-ranking score.

More specifically, for each object in the first constellation of objects, the computer system can: extract a first location; detect a first object type of the object (e.g., hands of human, tool, a cart containing surgical material, a lap sponge); derive a first surgical status of the object (e.g., hands of human walking from prep table toward operating table, a tool in hands of primary surgeon at operating table, a cart containing surgical material entering surgical space, a lap sponge falling within a threshold distance of and moving toward the operating table, a lap sponge in contact with and/or moving away from a patient on the operating table); calculate a first ranking score of the object based on the first object type and the first surgical status (e.g., risk of injury to surgical staff, risk of loss in the patient, object type, object proximity to surgical staff, object proximity to the patient, and/or relevance of the object to a current stage or step of the surgical operation); and store the first location, the first object type, the first surgical status, and the first ranking score in an object container in a set of object containers.

From this set of ranked objects, the computer system selects a first, highest-ranking object that intersects the cone of actuation of the mobile sensor unit. From the remaining set of ranked objects, the computer system then selects a second, highest-ranking object that intersects the cone of actuation of the wall-mounted dynamic camera. In particular, the computer system can: select a first target object, in the first constellation of objects, at a first time based on a first target ranking score of the first target object; and articulate the mobile camera to locate a first target location of the first target object in a field of view of the mobile camera. Then, the computer system can: select a second target object, in the first constellation of objects, at a second time based on a second target ranking score—greater than the first target ranking score—of the second target object; and articulate the mobile camera to locate a second target location of the second target object in the field of view of the mobile camera.

### 10.2 Optical Resolution

In one implementation, the computer system can execute Blocks of the method S100 to autonomously switch between tracking individual target objects (e.g., small, high-risk objects) moving within the surgical space at a low(er) resolution via images captured by the set of fixed optical sensors and tracking these individual target objects at a high(er) resolution via images captured by the mobile sensor (e.g., wall-mounted dynamic camera, mobile camera within the mobile sensor unit).

More specifically, the computer system can trigger the mobile camera to capture high(er) resolution images of a target object—exhibiting a highest target ranking score—in the surgical space at a first optical resolution at a first time. Then, in response to the target ranking score of the target object decreasing, or the ranking score of a next target object exhibiting a higher target ranking score, or a surgical status update of the target object, the computer system can trigger the set of fixed optical sensors to capture low(er) resolution images of the target object moving through the surgical space at a second optical resolution less than the first optical resolution at a second time. Accordingly, at approximately the second time, the computer system can trigger the mobile camera to capture high(er) resolution images of the next target object at the first optical resolution. The computer system can then derive a trajectory for each target object moving through the surgical space annotated with locations, object types, surgical statuses, and ranking scores stored in object containers and link low(er) resolution images and high(er) resolution images to a corresponding segment of a trajectory for each target object.

For example, the computer system can: articulate the mobile camera, facing and configured to image the surgical space at a first optical resolution, to locate a first target location of a first target object in the field of view of the mobile camera; access a first sequence of images captured by the mobile camera at the first optical resolution; extract a first set of locations and a first set of times of the first target object from the first sequence of images; and store the first set of locations and the first set of times of the first target object in a first target object container in the set of object containers. Then, in response to a second target ranking score of the second target object exceeding the first target ranking score of the first target object, the computer system can articulate the set of fixed optical sensors, facing and configured to image the surgical space at a second optical resolution less than the first optical resolution, to locate a second target location of the first target object. The computer system can then: access a second sequence of images captured by the set of fixed optical sensors at the second optical resolution; extract a second set of locations and a second set of times of the first target object from the second sequence of images; store the second set of locations and the second set of times of the first target object in the first target object container in the set of object containers; and derive a first target object trajectory based on locations, times, object types, surgical statuses, and ranking scores stored in the first target object container in the set of object containers. Thus, the computer system can leverage the first object trajectory to provide real-time intra-operative feedback and/or post-operative feedback via the display, as further described below.

In one variation, the computer system can: trigger the mobile camera to capture high(er) resolution images of objects moving within a particular region of the surgical space (e.g., a high-activity region depicting the prep table) at the first optical resolution; and trigger the set of fixed optical sensors to capture low(er) resolution images of objects moving within a next particular region of the surgical space (e.g., a high-activity region depicting the operating table) at a second optical resolution less than the first optical resolution.

For example, the computer system can: articulate the mobile camera, configured to image a first region of the surgical space (e.g., a high activity region depicting the operating table) at a first optical resolution, to locate the first target location of the first target object in the field of view of the mobile camera; and articulate the set of fixed optical sensors, configured to image a second region of the surgical space (e.g., a high activity region depicting the prep table) at a second optical resolution less than the first optical resolution, to locate the second target location of the first target object at approximately the second time.

Therefore, the computer system can autonomously switch between tracking a target object via the mobile camera at a high(er) optical resolution and tracking the target object via the set of fixed optical sensors at a low(er) optical resolution based on the target ranking score of the target object. The computer system can then derive a trajectory for the target object annotated with locations, an object type, surgical statuses, and ranking scores stored in a first target object container, link these low(er) resolution images and high(er) resolution images to a corresponding segment of a trajectory for each target object, and present the trajectory to surgical staff working within the surgical space via the display.

### 10.3 Example: Needle Driver+Needle

In one example, the computer system can execute Blocks of the method S100 to articulate a mobile camera to track a first target object (e.g., a needle driver) and a second target object (e.g., a needle) moving through the surgical space.

In this example, the computer system can implement methods and techniques described above to: select a first target object (e.g., a needle driver) in the first constellation of objects, at the first time based on the first target ranking score of the needle driver; articulate the mobile camera to locate a first target location of the needle driver in the field of view of the mobile camera; identify a second location of a second target object (e.g., a needle), in the first constellation of objects, at a second time; detect proximity of the needle to the needle driver based on the first target location and the second location; and calculate a second target ranking score of the needle based on proximity of the needle to the needle driver at the second time. The computer system can: detect an initial set of immutable objects—such as an operating table, a patient located on the operating table, a prep table—fixed in the surgical space, in the three-dimensional representation; and extract a location of the patient located on the operating table, a location of the prep table, and a location of the operating table from the three-dimensional representation of the surgical space.

The computer system can then: calculate a distance between the second location of the needle and the location of the patient located on the operating table; and, in response to the first distance falling below a threshold distance, increase the second target ranking score of the needle. Alternatively, the computer system can: calculate proximity of the needle to the patient located on the operating table based on the second location of the needle and the location of the patient; and increase the second target ranking score of the needle based on the proximity of the needle to the patient located on the operating table.

Accordingly, the computer system can: select the needle, in the first constellation of objects, at a third time based on the increased second target ranking score of the needle; articulate the mobile camera to locate a second target location of the needle in the field of view of the mobile camera; and derive a set of object trajectories based on locations, times, object types, surgical statuses, and ranking scores stored in the set of object containers.

### 10.4 Example: Lap Sponge+Needle Driver

In one example, the computer system can execute Blocks of the method S100 to articulate a mobile camera to track a first target object (e.g., a lap sponge, a surgical sponge) and a second target object (e.g., a loaded needle driver) moving through the surgical space.

In this example, the computer system can implement methods and techniques described above to identify a location of a first target object (e.g., a surgical sponge, lap sponge) in the first constellation of objects, at an initial time prior to the first time; detect proximity of the surgical sponge to a member of the surgical staff within the surgical space; and calculate a first target ranking score of the surgical sponge based on proximity of the surgical sponge to the member of surgical staff at the initial time. The computer system can then: select the surgical sponge, in the first constellation of objects, at the first time based on the first target ranking score of the surgical sponge; and articulate the mobile camera to locate the first target location of the surgical sponge in the field of view of the mobile camera.

Then, the computer system can: identify a second location of the second target object (e.g., a loaded needle driver), in the first constellation of objects, at a second time; detect proximity of the loaded needle driver to the operating table based on the second location and the location of the operating table within the surgical space; calculate a second target ranking score of the loaded needle driver based on proximity of the needle driver to the operating table at the second time; select the loaded needle driver, in the first constellation of objects, at a third time based on the second target ranking score of the loaded needle driver; and articulate the mobile camera to locate a second target location of the loaded needle driver in the field of view of the mobile camera.

### 11. System Modification

In one variation, the computer system can execute the foregoing methods and techniques to track the field of view of a single mobile camera (e.g., wall-mounted dynamic camera) to locate a target object in the surgical space. For example, the system 100 can be outfitted with a single wall-mounted dynamic camera for surgical operations involving a single primary surgeon or for low- to moderate-complexity surgical operations. Accordingly, the computer system can execute the method to track the single dynamic camera to target objects (e.g., sutures, sponges, tools) entering and exiting the surgeon's work envelope.

However, for surgical operations involving two surgeons and/or for higher-complexity surgical operations, an additional penetration mount and a second dynamic camera can be installed on the truss system in the surgical space, or an extant depth sensor installed on the truss system can be exchanged for a second wall-mounted dynamic camera. The computer system can then independently: control and process images output by the first dynamic camera to track objects entering and exiting a first surgeon's work envelope; and control and process images output by the second dynamic camera to track objects entering and exiting a second surgeon's work envelope.

### 12. Real-Time Interventions

The computer system can serve real-time feedback to surgical staff during a surgical operation such as: to re-register a target object in the field of view of mobile camera and/or an optical sensor when the computer system loses sight of the target object (hereinafter a “loss of sight event”); and to relocate the mobile sensor unit to reduce object loss events, as shown in FIG. 2.

Furthermore, the computer system can: calculate a target orientation of the mobile camera to locate a target location of a target object within the surgical space; prompt surgical staff to manually reposition the mobile camera; and prompt surgical staff to present a target object within the field of view of the mobile camera. For example, the computer system can: calculate a first target orientation of the mobile camera to locate a first target location of a first target object (e.g., a needle driver) in the field of view of the mobile camera; and generate a first prompt for a member of surgical staff to reposition the mobile camera within the surgical space to the target orientation. Then, in response to detecting absence of a second target object (e.g., a needle) in the field of view of the mobile camera, the computer system can: generate a second prompt for the member of surgical staff to present the second target object within the field of view of the mobile camera; extract a second location and a second surgical status of the second target object from the three-dimensional representation of the surgical space; and store the second location and the second surgical status of the second target object in a second object container in the set of object containers.

Alternatively, the computer system can autonomously articulate (e.g., reorient) a mobile camera and/or fixed optical sensor to a next location to track the target object in the field of view of the mobile camera and/or the fixed optical sensor.

More specifically, the computer system can implement methods and techniques described above to track a first target object and a second target object moving through the surgical space. Then, in response to detecting absence of a first target object in the field of view of the mobile camera, the computer system can: articulate the mobile camera to locate a third target location of the first target object in the field of view of the mobile camera; and, in response to detecting absence of the second target object in the field of view of the mobile camera, articulate the mobile camera to locate a fourth target location of the second target object in the field of view of the mobile camera.

### 12.1 Object Loss

Generally, the computer system can execute methods and techniques described above to: generate a sequence of three-dimensional maps of the surgical space based on sequences of depth images captured by the depth sensors; detect and track objects within these three-dimensional maps of the surgical space; and identify particular target objects within these three-dimensional maps; and autonomously navigate the mobile camera (e.g., wall-mounted dynamic camera and/or a mobile camera in the mobile sensor unit) to track individual target objects (especially small, high-risk objects, such as surgical needs, needle drivers, and surgical sponges) at greater resolution.

However, under various scenarios, a target object may be obfuscated and not visible in the field of view of the wall-mounted dynamic camera or the mobile sensor unit currently tracking the target object and/or in the fields of view of the depth sensors (hereinafter “loss of sight events”).

In one implementation, the computer system can selectively generate a prompt to bring a target object back into the field of view of the mobile sensor unit or wall-mounted dynamic camera and present this prompt to surgical staff, such as by rendering this prompt on the display.

For example, a surgical staff member returning a target object (e.g., a surgical sponge, a needle) from the surgical table to the prep table may: stand between the wall-mounted dynamic camera and the target object; and then place the target object in a container (e.g., a biowaste container) before the computer system detects the target object in the field of view of the wall-mounted dynamic camera. In this example, the computer system can generate a prompt to move the target object back into the field of view of the wall-mounted dynamic camera (or the mobile sensor unit); populate the prompt with a descriptor (e.g., a type) of the target object and a last detected location of the target object within the surgical space; and serve the prompt to surgical staff, such as by rendering the prompt on the display and outputting an audible alarm.

The computer system can then implement methods and techniques described above to: generate a next three-dimensional map of the surgical space based on a next set of depth images captured by the depth sensors; scan the three-dimensional map for the target object; access a next 2D image captured by the wall-mounted dynamic camera (or the mobile sensor unit); and scan this 2D image for the target object. Once the target object is detected in the three-dimensional map or the surgical space and/or the 2D image, the computer system can: reregister motion of the wall-mounted dynamic camera (or the mobile sensor unit) to the target object; and return confirmation to surgical staff that the target object was re-registered.

In another example, small target objects (e.g., a surgical needle) on the prep table may be directly visible in the field of view of the wall-mounted dynamic camera and/or the mobile sensor unit when a surgical staff member is not present at the prep table or handing the target object. Accordingly, the computer system can directly track a small target object in images captured by the wall-mounted dynamic camera and/or the mobile sensor unit. However, when the surgical staff member retrieves the target object, the target object may be obscured, such as by a hand or a needle driver grasping the target object. Therefore, the computer system can: transition to tracking the hand or needle driver as a proxy for the location of the target object in the surgical space; and then transition back to tracking the target object directly once the target object leaves the hand or needle driver.

However, once the computer system transitions to tracking the hand or needle driver as a proxy for the location of the target object in the surgical space, the computer system can generate a prompt to move the target object back into the field of view of the wall-mounted dynamic camera or mobile sensor unit, such as: a) if the computer system detects the hand or needle driver grasping or engaging another object before the computer system re-registers the target object in the field of view of the wall-mounted dynamic camera or mobile sensor unit; or b) if more than a threshold time (e.g., two minutes for a needle, one minute for a surgical sponge) elapses before the computer system re-registers the target object in the field of view of the wall-mounted dynamic camera or mobile sensor unit.

In another example, surgical staff may move through the surgical space with target objects during the surgical operation and may: a) inadvertently move a current target object out of the cone of actuation of the wall-mounted dynamic camera; and/or b) move the current target object to a location in the surgical space that is too far from the wall-mounted dynamic camera to image with sufficient resolution for the computer system to confidently identify the target object. Accordingly, the computer system can generate a prompt: to move the target object back into the field of view of the wall-mounted dynamic camera or mobile sensor unit; and to avoid moving the target object and/or other objects in the future along the same trajectory.

### 12.1.1 Probability of Loss

In one implementation, the computer system can implement methods and techniques described above to track a first target object and, in response to detecting absence of the first target object within the field of view of the mobile camera, predict a probability of loss (e.g., surgical object left behind in patient) of the first target object.

For example, the computer system can: identify a first location of a first target object (e.g., a surgical sponge) at an initial time; detect proximity of the surgical sponge to a patient located on the operating table at a second location within the surgical space; and calculate a first target ranking score of the surgical sponge based on proximity of the surgical sponge to the patient located on the operating table at the initial time based on the first location and the second location. The computer system can then: select the surgical sponge at the first time based on the first target ranking score of the surgical sponge; and articulate the mobile camera to locate a first target location of the surgical sponge in the field of view of the mobile camera. Then, the computer system can: in response to detecting absence of the first target object in the field of view of the mobile camera, predict a probability of loss of the surgical sponge inside the patient located on the operating table; and, in response to the probability of loss exceeding a probability of loss threshold, issue an alarm for manual survey of surgical sponges in the surgical space and serve a prompt, to surgical staff in the surgical space, to return a quantity of surgical sponges to a disposal container (e.g., biowaste container).

### 12.2 Mobile Sensor Unit Relocation

Additionally or alternatively, the computer system can generate a prompt to relocate the mobile sensor unit in order to decrease frequency of target object loss of sight events.

In one implementation, the computer system: executes the foregoing methods and techniques to identify target object loss of sight events; stores last known locations of target objects tracked by the mobile sensor unit before target object loss of sight events; and stores locations or trajectories of all target objects tracked by the mobile sensor unit during the surgical operation.

Once the quantity or frequency of target object loss of sight events at the mobile sensor unit exceeds a threshold quantity (e.g., four) or a threshold frequency (e.g., five per hour), the computer system can calculate a new location of the mobile sensor unit: that yields a cone of actuation of the mobile sensor unit that encompasses the last known locations of target objects before target object loss of sight events; that yields a cone of actuation of the mobile sensor unit that encompasses locations or trajectories of all target objects tracked by the mobile sensor unit during the surgical operation; and that locates the last known locations of target objects before target object loss of sight events at different positions within the cone of actuation—such that the mobile sensor unit can view these locations from different perspectives that may produce fewer target object loss of sight events.

The computer system can then serve a prompt to relocate the mobile sensor unit to this new location, such as a prompt to: raise or lower the dynamic camera on the mast of the mobile sensor unit; and/or move (or “wheel”) the mobile sensor unit to a different target floor location within the surgical space.

The computer system can then implement methods and techniques described above to recalibrate the location and/or the field of view model of the mobile sensor unit after detecting relocation of the mobile sensor unit.

### 13. Sensor Position Feedback

The computer system can also serve pre-operative guidance and post-operative feedback for mobile sensor unit positioning within the surgical space.

### 13.1 Pre-Operative Feedback

In one variation, during or upon conclusion of a surgical operation, the computer system generates a mobile sensor unit record containing: timeseries positions of the mobile sensor unit throughout the surgical operation; and characteristics of the surgical operation, such as including surgery type, primary surgeon and/or other surgical staff, hospital clinic, surgery duration, steps of the surgical operation, objects present in the surgical space during the surgical operation, objects tracked during the surgical operation, and/or types and frequency of loss of sight events. The computer system can write this surgical operation record to a surgery database.

In preparation for a next surgical operation in the surgical space, the computer system (or other device or computer network) can retrieve characteristics of this next surgical operation, such as including: type of surgical operation; primary surgeon and/or surgical staff; surgeon preferences for object tracking; and/or clinical surgical record and object tracking requirements. The computer system can then: search the surgery database for a past surgical operation with similar characteristics; and extract a mobile sensor unit position—exhibiting lowest frequency of loss of sight events in this past surgical operation—from a particular record of this past surgical operation best matched to characteristics of the next surgical operation. Accordingly, the computer system can generate a recommendation to initially locate the mobile sensor unit at this position in the surgical space at the start of the surgical operation.

For example, the computer system can access a surgical record from a surgery database, the surgical record defining: a type of surgery; a primary surgeon associated with the type of surgery; a duration of the type of surgery; and an initial set of trajectories of an initial constellation of objects for the type of surgery. The computer system can then extract a set of initial mobile camera positions from the initial set of trajectories of the initial constellation of objects for the type of surgery. Accordingly, computer system can identify similar characteristics between this initial type of surgery and a next surgical operation and implement methods and techniques described above for the next surgical operation. The computer system can then: predict a first target position of the mobile camera based on the set of initial mobile camera positions; and articulate the mobile camera to a first target position to locate a first target location of a first target object (e.g., a needle driver) in the field of view of the mobile camera; predict a second target position of the mobile camera based on the set of initial mobile camera positions; and articulate the mobile camera to the second target position to locate the second target location of a second target object (e.g., a needle) in the field of view of the mobile camera.

### 13.2 Intra-Operative Feedback

Furthermore, during the next surgical operation, the computer system can derive trajectories for a constellation of objects including the first target object and the second target object, characterize a difference between the initial set of trajectories and these trajectories, and provide real-time feedback to surgical staff indicating this difference.

In one implementation, the computer system can: derive trajectories for a first constellation of objects—including a first target object (e.g., a needle driver) and a second target object (e.g., a needle)—moving within the surgical space; and characterize a difference between the initial set of trajectories of the initial constellation of objects and the first set of trajectories of the first constellation of objects. Then, in response to the difference between the initial set of trajectories of the initial constellation of objects and the first set of trajectories of the first constellation of objects exceeding a difference threshold, the computer system can: generate a notification alerting surgical staff within the surgical space of the difference; and transmit the notification to the display for review by surgical staff.

### 13.3 Post-Operative Feedback

Additionally or alternatively, the computer system can generate mobile sensor unit metrics and statistics for mobile sensor unit deployment during a past or current surgical operation, such as: instances that a target object was lost from view during the surgical operation; instances that a target object was tracked with less than a minimum resolution or minimum confidence score in images captured by the wall-mounted dynamic camera; number of instances or durations in which the computer system identified multiple target objects that are not all concurrently visible in the field of view of the wall-mounted dynamic camera at sufficient resolution for tracking (e.g., a needle driver passing from a prep table toward an operating table while a lap sponge moves into and out of the patient); number of instances that the computer system prompted surgical staff to move the mobile sensor unit; delay time between prompts to move the mobile sensor unit and movement of the mobile sensor unit; and a representation of overlap between fields of view of the depth sensors over time throughout the surgery (e.g., an integral of overlap of depth sensor fields of view over time during the surgery, a minimum overlap of fields of view of the sensors, or an average overlap of fields of view of the sensors).

In one implementation, based on these metrics, the computer system can generate a prompt or recommendation to add a mobile sensor unit in the surgical space, such as responsive to: an excess quantity or excess rate of loss of sight events occurring in the surgical operation during the surgical operation; or high latency between prompting surgical staff to move the (single) mobile sensor unit in the surgical space and corresponding relocation of the mobile sensor unit.

Similarly, based on these metrics, the computer system can generate a prompt or recommendation to add a wall-mounted dynamic camera in the surgical space, such as responsive to: excess quantity or excess rate of instances in which the computer system identifies more target objects necessitating tracking than wall-mounted dynamic cameras available in the surgical space.

The systems and methods described herein can be embodied and/or implemented at least in part as a machine configured to receive a computer-readable medium storing computer-readable instructions. The instructions can be executed by computer-executable components integrated with the application, applet, host, server, network, website, communication service, communication interface, hardware/firmware/software elements of a user computer or mobile device, wristband, smartphone, or any suitable combination thereof. Other systems and methods of the embodiment can be embodied and/or implemented at least in part as a machine configured to receive a computer-readable medium storing computer-readable instructions. The instructions can be executed by computer-executable components integrated by computer-executable components integrated with apparatuses and networks of the type described above. The computer-readable medium can be stored on any suitable computer readable media such as RAMs, ROMs, flash memory, EEPROMs, optical devices (CD or DVD), hard drives, floppy drives, or any suitable device. The computer-executable component can be a processor but any suitable dedicated hardware device can (alternatively or additionally) execute the instructions.

As a person skilled in the art will recognize from the previous detailed description and from the figures and claims, modifications and changes can be made to the embodiments of the invention without departing from the scope of this invention as defined in the following claims.

I claim:

1. A method for surgical object imaging within a surgical space comprising:
accessing a first set of images captured by a set of fixed optical sensors arranged about and facing the surgical space;
aggregating the first set of images into a three-dimensional representation of the surgical space;
detecting a first constellation of objects, moving within the surgical space, in the three-dimensional representation;
based on the three-dimensional representation of the surgical space, for each object in the first constellation of objects:
extracting a first location of the object;
detecting a first object type of the object;
deriving a first surgical status of the object;
calculating a first ranking score for the object based on the first object type and the first surgical status; and
storing the first location, the first object type, the first surgical status, and the first ranking score in an object container in a set of object containers;
selecting a first target object, in the first constellation of objects, at a first time based on a first target ranking score of the first target object;
articulating a mobile camera to locate a first target location of the first target object in a field of view of the mobile camera;
selecting a second target object, in the first constellation of objects, at a second time succeeding the first time based on a second target ranking score of the second target object, the second target ranking score greater than the first target ranking score of the first target object;
articulating the mobile camera to locate a second target location of the second target object in the field of view of the mobile camera; and
deriving a set of trajectories of the first constellation of objects based on object types, locations, surgical statuses, and ranking scores stored in the set of object containers.

2. The method of claim 1, further comprising:
in response to detecting absence of the first target object in the field of view of the mobile camera at a third time succeeding the first time, articulating the mobile camera to locate a third target location of the first target object in the field of view of the mobile camera; and
in response to detecting absence of the second target object in the field of view of the mobile camera at a fourth time succeeding the second time, articulating the mobile camera to locate a fourth target location of the second target object in the field of view of the mobile camera.

3. The method of claim 1:
wherein articulating the mobile camera to locate the first target location of the first target object comprises:
calculating a first target orientation of the mobile camera to locate the first target location of the first target object in the field of view of the mobile camera; and
generating a first prompt for a member of surgical staff to reposition the mobile camera within the surgical space to the target orientation; and
further comprising, in response to detecting absence of the second target object in the field of view of the mobile camera at a third time succeeding the second time:
generating a second prompt for the member of surgical staff to present the second target object within the field of view of the mobile camera;
extracting a second location and a second surgical status of the second target object from the three-dimensional representation of the surgical space; and
storing the second location and the second surgical status of the second target object in a second object container in the set of object containers.

4. The method of claim 1:
further comprising, during a setup period:
accessing an initial set of images captured by the set of fixed optical sensors arranged about and facing the surgical space;
detecting a set of overlapping features between the initial set of images;
deriving a set of transforms based on the set of overlapping features between the initial set of images; and
applying the set of transforms to the initial set of images to assemble an initial three-dimensional map in a set of three-dimensional maps of the surgical space; and
wherein aggregating the first set of images into the three-dimensional representation of the surgical space comprises:
applying the set of transforms to the first set of images to assemble a first three-dimensional map in the set of three-dimensional maps of the surgical space; and
combining the set of three-dimensional maps of the surgical space into the three-dimensional representation of the surgical space.

5. The method of claim 1:
wherein selecting the first target object, in the first constellation of objects, comprises selecting the first target object comprising a needle driver, in the first constellation of objects, at the first time based on the first target ranking score of the needle driver;
wherein articulating the mobile camera to locate the first target location of the first target object comprises articulating the mobile camera to locate the first target location of the needle driver in the field of view of the mobile camera;
further comprising:
identifying a second location of the second target object comprising a needle, in the first constellation of objects, at a third time between the first time and the second time;
detecting proximity of the needle to the needle driver based on the first target location and the second location; and
calculating the second target ranking score of the needle based on proximity of the needle to the needle driver at the third time;
wherein selecting the second target object, in the first constellation of objects, comprises selecting the needle, in the first constellation of objects, at the second time based on the second target ranking score of the needle; and
wherein articulating the mobile camera to locate the second target location of the second target object comprises articulating the mobile camera to locate the second target location of the needle in the field of view of the mobile camera.

6. The method of claim 5, further comprising:
detecting an initial set of immutable objects, fixed in the surgical space, in the three-dimensional representation of the surgical space at an initial time, the initial set of immutable objects comprising a patient located on an operating table; and
extracting a third location of the patient located on the operating table from the three-dimensional representation of the surgical space; and
calculating a first distance between the second location of the needle and the third location of the patient located on the operating table at a third time; and
in response to the first distance falling below a threshold distance, increasing the second target ranking score of the needle.

7. The method of claim 1, further comprising:
detecting an initial set of immutable objects, fixed in the surgical space, in the three-dimensional representation of the surgical space at an initial time, the initial set of immutable objects comprising:
a prep table;
an operating table; and
a patient located on the operating table; and
extracting a second location of the prep table, a third location of the operating table, and a fourth location of the patient located on the operating table from the three-dimensional representation of the surgical space.

8. The method of claim 7:
further comprising:
identifying a fifth location of the first target object comprising a surgical sponge, in the first constellation of objects, at the initial time prior to the first time;
detecting proximity of the surgical sponge to a member of surgical staff within the surgical space; and
calculating the first target ranking score of the surgical sponge based on proximity of the surgical sponge to the member of surgical staff at the initial time;
wherein selecting the first target object, in the first constellation of objects, comprises selecting the surgical sponge, in the first constellation of objects, at the first time based on the first target ranking score of the surgical sponge;
wherein articulating the mobile camera to locate the first target location of the first target object comprises articulating the mobile camera to locate the first target location of the surgical sponge in the field of view of the mobile camera;
further comprising:
identifying a sixth location of the second target object comprising a loaded needle driver, in the first constellation of objects, at a third time between the first time and the second time;
detecting proximity of the loaded needle driver to the operating table based on the sixth location and the third location; and
calculating the second target ranking score of the loaded needle driver based on proximity of the needle driver to the operating table at the third time;
wherein selecting the second target object, in the first constellation of objects, comprises selecting the loaded needle driver, in the first constellation of objects, at the second time based on the second target ranking score of the loaded needle driver; and
wherein articulating the mobile camera to locate the second target location of the second target object comprises articulating the mobile camera to locate the second target location of the loaded needle driver in the field of view of the mobile camera.

9. The method of claim 1:
further comprising:
identifying a second location of the first target object comprising a surgical sponge, in the first constellation of objects, at an initial time prior to the first time;
detecting proximity of the surgical sponge to a patient located on the operating table at a third location within the surgical space; and
calculating the first target ranking score of the surgical sponge based on proximity of the surgical sponge to the patient located on the operating table at the initial time based on the second location and the third location;
wherein selecting the first target object, in the first constellation of objects, at the first time comprises selecting the first target object comprising the surgical sponge, in the first constellation of objects, at the first time based on the first target ranking score of the surgical sponge;
wherein articulating the mobile camera to locate the first target location of the first target object comprises articulating the mobile camera to locate the first target location of the surgical sponge in the field of view of the mobile camera; and
further comprising, in response to detecting absence of the first target object in the field of view of the mobile camera at a third time between the first time and the second time:
predicting a probability of loss of the surgical sponge inside the patient located on the operating table; and
in response to the probability of loss exceeding a probability of loss threshold:
issuing an alarm for manual survey of surgical sponges in the surgical space; and
serving a prompt, to surgical staff in the surgical space, to return a quantity of surgical sponges to a disposal container.

10. The method of claim 1:
further comprising:
accessing a surgical record from a surgery database at an initial time preceding the first time, the surgical record defining:
a type of surgery;
a primary surgeon associated with the type of surgery;
a duration of the type of surgery; and
an initial set of trajectories of an initial constellation of objects for the type of surgery; and
extracting a set of initial mobile camera positions from the initial set of trajectories of the initial constellation of objects for the type of surgery;
wherein articulating the mobile camera to locate the first target location of the first target object comprises:
predicting a first target position of the mobile camera based on the set of initial mobile camera positions; and
articulating the mobile camera to the first target position to locate the first target location of the first target object in the field of view of the mobile camera; and
wherein articulating the mobile camera to locate the second target location of the second target object comprises:
predicting a second target position of the mobile camera based on the set of initial mobile camera positions; and
articulating the mobile camera to the second target position to locate the second target location of the second target object in the field of view of the mobile camera.

11. The method of claim 10, further comprising:
characterizing a difference between the initial set of trajectories of the initial constellation of objects and the first set of trajectories of the first constellation of objects at a third time; and
in response to the difference between the initial set of trajectories of the initial constellation of objects and the first set of trajectories of the first constellation of objects exceeding a difference threshold:
generating a notification alerting surgical staff within the surgical space of the difference; and
transmitting the notification to surgical staff.

12. The method of claim 1:
wherein articulating the mobile camera to locate the first target object comprises articulating the mobile camera, facing and configured to image the surgical space at a first optical resolution, to locate the first target location of the first target object in the field of view of the mobile camera; and
further comprising:
accessing a first sequence of images captured by the mobile camera at the first optical resolution at a third time;
extracting a first set of locations and a first set of times of the first target object from the first sequence of images; and
storing the first set of locations and the first set of times of the first target object in a first target object container in the set of object containers.

13. The method of claim 12:
wherein selecting the second target object, in the first constellation of objects, comprises in response to the second target ranking score of the second target object exceeding the first target ranking score of the first target object, articulating the set of fixed optical sensors, facing and configured to image the surgical space at a second optical resolution less than the first optical resolution, to locate a third target location of the first target object at approximately the second time;
further comprising:
accessing a second sequence of images captured by the set of fixed optical sensors at the second optical resolution at a fourth time;
extracting a second set of locations and a second set of times of the first target object from the second sequence of images; and
storing the second set of locations and the second set of times of the first target object in the first target object container in the set of object containers; and
wherein deriving the set of trajectories of the first constellation of objects comprises deriving a first target object trajectory in the set of trajectories based on locations, times, object types, surgical statuses, and ranking scores stored in the first target object container in the set of object containers.

14. A method for surgical object imaging within a surgical space comprising:
during a first time period:
accessing a three-dimensional representation of the surgical space;
based on the three-dimensional representation of the surgical space, for each object in a first constellation of objects moving within the surgical space:
extracting a first location of the object;
detecting a first object type of the object;
deriving a first surgical status of the object;
calculating a first ranking score for the object based on the first object type and the first surgical status; and
storing the first location, the first object type, the first surgical status, and the first ranking score in an object container in a set of object containers;
selecting a needle driver, in the first constellation of objects, based on a first target ranking score of the needle driver; and
articulating a mobile camera to locate a first target location of the needle driver in a field of view of the mobile camera; and
during a second time period succeeding the first time period:
identifying a second location of a needle, in the first constellation of objects;
detecting proximity of the needle to the needle driver based on the first target location and the second location;
calculating a second target ranking score of the needle based on proximity of the needle to the needle driver at the third time; and
in response to the second target ranking score of the needle exceeding the first target ranking score of the needle driver:
selecting the needle, in the first constellation of objects, at a third time based on the second target ranking score; and
articulating the mobile camera to locate a second target location of the needle in the field of view of the mobile camera.

15. The method of claim 14:
further comprising, during the first time period:
detecting an initial set of immutable objects, fixed in the surgical space, in the three-dimensional representation of the surgical space, the initial set of immutable objects comprising a patient located on an operating table; and
extracting a third location of the patient located on the operating table from the three-dimensional representation of the surgical space; and
further comprising, during the second time period:
calculating proximity of the needle to the patient located on the operating table based on the second location and the third location; and
increasing the second target ranking score of the needle based on the proximity of the needle to the patient located on the operating table.

16. The method of claim 14:
further comprising during the first time period:
accessing a surgical record from a surgery database, the surgical record defining an initial set of trajectories of an initial constellation of objects for the type of surgery; and
extracting a set of initial mobile camera positions from the initial set of trajectories of the initial constellation of objects for the type of surgery; and
wherein articulating the mobile camera to locate the first target location of the needle driver comprises:
predicting a first target position of the mobile camera based on the set of initial mobile camera positions; and
articulating the mobile camera to the first target position to locate the first target location of the needle driver in the field of view of the mobile camera.

17. The method of claim 14, wherein articulating the mobile camera to locate the first target location of the needle driver comprises:
calculating a first target orientation of the mobile camera to locate the first target location of the needle driver in the field of view of the mobile camera; and
generating a first prompt for a member of surgical staff to reposition the mobile camera within the surgical space to the first target orientation.

18. The method of claim 14, further comprising deriving a set of object trajectories based on locations, times, object types, surgical statuses, and ranking scores stored in the set of object containers.

19. A method for surgical object imaging within a surgical space comprising:
accessing a three-dimensional representation of the surgical space;
based on the three-dimensional representation of the surgical space, for each object in a first constellation of objects moving within the surgical space:
extracting a first location of the object;
detecting a first object type of the object;
deriving a first surgical status of the object;
calculating a first ranking score for the object based on the first object type and the first surgical status; and
storing the first location, the first object type, the first surgical status, and the first ranking score in an object container in a set of object containers;
selecting a first target object, in the first constellation of objects, at a first time based on a first target ranking score of the first target object;
articulating a mobile camera to locate a first target location of the first target object in the field of view of the mobile camera;
in response to detecting absence of the first target object within the field of view of the mobile camera at a second time, articulating a set of fixed optical sensors, arranged about and facing the surgical space, to locate a second target location of the first target object at approximately the second time;
in response to detecting presence of the first target object within the field of view of the mobile camera at a third time, articulating the mobile camera to locate a third target location of the first target object at approximately the third time; and
deriving a trajectory of the first target object in a set of trajectories based on images captured by the mobile camera and the set of fixed optical sensors.

20. The method of claim 19:
wherein articulating the mobile camera to locate the first target location comprises articulating the mobile camera, configured to image a first region of the surgical space at a first optical resolution, to locate the first target location of the first target object in the field of view of the mobile camera; and
wherein articulating the set of fixed optical sensors comprises articulating the set of fixed optical sensors, configured to image a second region of the surgical space at a second optical resolution less than the first optical resolution, to locate the second target location of the first target object at approximately the second time.
